(19) United States
(12) Patent Application Publication
(10) Pub. No.: US 2025/0082800 A1
(43) Pub. Date: Mar. 13, 2025

(54) SYSTEMS FOR IMPROVED BACTERIAL AND MICROBIAL REDUCTION, EQUIPMENT COMPRISING SAID SYSTEMS AND METHODS OF UTILIZING SAID SYSTEMS AND EQUIPMENT TO IMPROVE BACTERIAL AND MICROBIAL REDUCTION ON FOODS, FOOD PRODUCTS, AND PRODUCE

(71) Applicant: CleanWorks Inc., St. Catharines (CA)

(72) Inventors: Mark VANDERVEEN, St. Catharines (CA); Luke COURT, St. Catharines (CA); John PETITTI, St. Catharines (CA)

(73) Assignee: CleanWorks Inc., St. Catharines (CA)

(21) Appl. No.: 18/826,651

(22) Filed: Sep. 6, 2024

Related U.S. Application Data
(60) Provisional application No. 63/537,157, filed on Sep. 7, 2023.

(51) Int. Cl. A61L 2/10; A61L 2/18; A61L 2/20

(52) U.S. Cl. CPC A61L 2/10; A61L 2/183; A61L 2/202; A61L 2202/11; A61L 2202/15

(57) ABSTRACT

Systems and methods treat produce on vines or in fields with hydroxyl-radical treatments and provide antimicrobial hydroxyl-radicals of the hydroxyl-radical treatments by exposing hydrogen peroxide mist and/or ozone gas within a process chamber to UV-C light and/or radiation emitted by one or more UV-C lamps. The methods treat the produce on the vines or in the fields with the one or more hydroxyl-radicals of the hydroxyl-radical treatments by moving the systems over, above, and/or around the produce on the vines or in the fields. The systems have a movable frame, at least one mist producing unit in fluid communication with the processing chamber, one or more UV-C lamps disposed within or adjacent to the processing chamber, and at least one ozone gas producing unit in fluid communication with the processing chamber.

The present disclosure relates to and/or is directed to systems, equipment, and/or methods configured/adapted for improving bacterial and/or microbial reduction and/or providing pathogen decontamination of foods, food products, plants, and/or produce. The systems and/or methods disclosed herein may be incorporated into and/or included within or attached, connected, or coupled to equipment for improving bacterial and/or microbial reduction and/or providing improved pathogen decontamination of foods, food products, plants, and/or produce. The systems and equipment disclosed herein may be utilized by one or methods for treating foods, food products, plants, and/or produce. As a result of the treatment disclosed herein, improved bacterial and/or microbial reduction and/or improved pathogen decontamination of foods, food products, plants, and/or produce are achievable by the systems, equipment, and/or methods disclosed herein. The systems, equipment, and/or methods disclosed herein may generate, provide, and/or produce hydroxyl-radicals for treating foods, food products, plants, vines, and/or produce that may be grown or maintained in and/or harvested from one or more farm fields. As a result of the hydroxyl-radical treatment disclosed herein, improved bacterial and/or microbial reduction and/or improved pathogen decontamination of the foods, food products, plants, vines, and produce grown or maintained in and/or harvested from the one or more farm fields is achievable. The present systems, equipment, and/or methods disclosed herein may generate the hydroxyl-radicals by introducing a misting of a low concentration hydrogen peroxide mist into a treatment chamber containing one or more UV-C lamps with or in addition to ozone gas being present in or disposed within the treatment chamber.

### SUMMARY OF THE DISCLOSURE

In one or more embodiments, systems, equipment, and/or methods may be configured to or adapted to treat vines, plants, and/or produce on at least one vine and/or in at least one farm field with a hydroxyl-radical treatment and/or may expose the vines, plants, and/or produce to one or more hydroxyl-radical treatments. In some embodiments, the systems, equipment, and/or methods may comprise at least one processing or treatment chamber and/or at least one mist producing unit disposed within or adjacent to the processing or treatment chamber for introducing hydrogen peroxide mist therein. The systems, equipment, and/or methods may further comprise at least one UV-C lamp unit disposed within or adjacent to the processing or treatment chamber and at least one ozone gas producing unit disposed within or adjacent to the processing or treatment chamber. The processing or treatment chamber may be movable over and/or along the vines, plants, and/or produce such that improved bacterial and/or microbial reduction and/or improved pathogen decontamination of the vines, plants, and/or produce may be achievable via the hydroxyl-radical treatment emitted, produced, provided, and/or disposed within the processing or treatment chamber.

In some embodiments, treatment equipment may generate, produce, and/or provide the hydroxyl-radical treatment and/or may comprise at least one movable vehicle with the present treatment equipment attached, connected, and/or coupled to the at least one movable vehicle. In some embodiments, the at least one movable vehicle and the present treatment equipment may be configured for or adapted to move above or along the vines, plants, and/or produce on the at least one vine or in the at least one farm field such that the vines, plants, and/or produce may pass through the processing or treatment chamber or at least a part of the processing or treatment chamber of the present treatment equipment. As a result, the vines, plants, and/or produce on the at least one vine or in the at least one field may be exposed to the hydroxyl-radical treatment provided within the processing or treatment chamber and improved bacterial and/or microbial reduction and/or improved pathogen decontamination of the vines, plants, and/or produce is achieved by the hydroxyl-radical treatment via the at least one movable vehicle and/or the present system or treatment equipment disclosed herein.

In an embodiment, wherein the movable vehicle may be a movable or drivable vehicle, a tractor, a harvester, a combine, a tow, movable farm equipment, or a combination thereof.

In one or more embodiments, the methods disclosed herein may comprise providing hydroxyl-radicals of a hydroxyl-radical treatment by introducing a misting of hydrogen peroxide mist into the processing or treatment chamber of present treatment equipment and/or systems, wherein the processing or treatment chamber contains the at least one UV-C lamp unit and, or in addition to, ozone gas provided by and/or produced or expelled from the at least one ozone gas producing unit. The present methods may further comprise treating the vines, plants, and/or produce on the at least one vine or in the at least one farm field with the hydroxyl-radical treatment by moving the present treatment equipment and/or systems over, above, and/or around the vines plants, and/or produce on the at least one vine or in the at least one farm field. As a result of moving the present treatment equipment and/or systems over, above, and/or around the vines, plants, and/or produce, improved bacterial and/or microbial reduction and/or improved pathogen decontamination of the vines, plants, and/or produce is achievable via the hydroxyl-radical treatment provided by the present treatment equipment and/or system, and/or the movable vehicle attached, connected, and/or coupled to the present treatment equipment and/or system.

In some embodiments, a system configure for treating produce on the vine or in the field with a hydroxyl-radical treatment is provided and may comprise a movable frame defining a processing chamber between a first end of the movable frame and a second end of the movable frame that is opposite with respect to the first end of the movable frame, at least one mist producing unit disposed connected to the movable frame and in fluid communication with the processing chamber for introducing hydrogen peroxide mist into the processing chamber, one or more UV-C lamps connected to the movable frame and disposed within or adjacent to the processing chamber for emitting UV-C light and/or radiation into the processing chamber, and at least one ozone gas producing unit connected to the movable frame and in fluid communication with the processing chamber for introducing ozone gas into the processing chamber, wherein the processing chamber is movable over and/or along the produce on the vine or in the field and antimicrobial hydroxy-radicals of the hydroxyl-radical treatment are formed via the hydrogen peroxide, UV-C light and/or radiation, and the ozone gas within the processing chamber such that bacterial and/or microbial reduction and/or pathogen decontamination of the produce is achievable via the antimicrobial hydroxy-radicals of the hydroxyl-radical treatment emitted within the processing chamber.

In an embodiment, the system may further comprise a cage connected to the movable frame, wherein the processing chamber is further defined by one or more surfaces of the cage.

In an embodiment, the system may further comprise one or more light panels comprising the one or more UV-C lamps and connected to the movable frame by the cage.

In an embodiment, the system may further comprise a first nozzle assembly connected to the movable frame at or adjacent to the first end of the movable frame such that nozzles of the first nozzle assembly are directed towards the processing chamber.

In an embodiment, the system may further comprise a second nozzle assembly connected to the movable frame at an intermediate position or location between the first and second ends of the movable frame such that nozzles of the second nozzle assembly are directed towards the processing chamber.

In an embodiment, the at least one of the at least one mist producing unit and the at least one ozone gas producing unit may be in fluid communication with the processing chamber via at least one of the first nozzle assembly and the second nozzle assembly.

In an embodiment, the one or more UV-C lamps may comprise first UV-C lamps and second UV-C lamps and the second UV-C lamps are angled at an angle with respect to the first UV-C lamps.

In an embodiment, the angle may be greater than about 0° and less than about 90°.

In an embodiment, the angle may range from about 30° to about 60°.

In an embodiment, the system may further comprise at least one air knife assembly connected to the movable frame, in fluid communication with the processing chamber, and configured to mix the ozone gas and the hydrogen peroxide mist to provide a treatment mixture within the processing chamber.

In an embodiment, the system may further comprise at least one nozzle assembly connected to the movable frame, disposed adjacent to the at least one air knife assembly, and in fluid communication with the at least one mist producing unit for introducing the hydrogen peroxide mist into the processing chamber.

In one or more embodiments, treatment equipment is provided and may comprise a movable vehicle and the present system disclosed herein connected or coupled to the movable vehicle.

In an embodiment, the movable vehicle may be selected from the group consisting of a tractor, a harvester, a tow, a trailer, and a wagon.

In an embodiment, the system may further comprise a vehicle propulsion system connected to the movable frame and configured for towing system with the movable vehicle.

In one or more embodiments, a method is provided and may comprise providing one or more antimicrobial hydroxyl-radicals of the hydroxyl-radical treatment by exposing the hydrogen peroxide mist and/or ozone gas within the process chamber to UV-C light and/or radiation emitted by the one or more UV-C lamps, and treating produce on a vine or in a field with the one or more hydroxyl-radicals of the hydroxyl-radical treatment by moving the system of claim 1 over, above, and/or around the produce on the vine or in the field.

In an embodiment, the hydrogen peroxide mist may comprise hydrogen peroxide at a concentration from about 0.1% to about 10%, based on a total weight of the hydrogen peroxide mist.

In an embodiment, a wavelength emitted by the one or more UV-C lamps may range from about 220 nm to about 280 nm.

In an embodiment, the wavelength emitted by the one or more UV-C lamps may be about 254 nm.

In an embodiment, the produce may be present within the processing chamber for a dwell time from about 1 second to about 15 seconds.

In an embodiment, the system may have a tracker speed with respect to the produce on the vine or in the field ranging from about 0.5 km/hr to about 7.5 km/hr.

### BRIEF SUMMARY OF THE DRAWINGS

The present disclosure is best understood from the following detailed description when read with the figures, illustrations, drawings, configurations, renders disclosed herein. It is emphasized that, in accordance with the standard practice in the industry, various features are not drawn to scale. In fact, the dimensions of the various features may be arbitrarily increased or reduced for clarity of discussion.

FIG. 1 illustrates a perspective view of a treatment system or equipment disclosed herein and configured/adapted for improving bacterial and/or microbial reduction and/or providing improved pathogen decontamination of vines, plants, and/or produce on at least one vine or in at least one farm field, in at least one embodiment of the present disclosure.

FIG. 2 illustrates another perspective view of a treatment system or equipment disclosed herein and configured/adapted for improving bacterial and/or microbial reduction and/or providing improved pathogen decontamination of vines, plants, and/or produce on at least one vine or in at least one farm field, in at least one embodiment of the present disclosure.

FIG. 3 illustrates a side plan view of a treatment system or equipment disclosed herein and configured/adapted for improving bacterial and/or microbial reduction and/or providing improved pathogen decontamination of vines, plants, and/or produce on at least one vine or in at least one farm field, in at least one embodiment of the present disclosure.

FIG. 4 illustrates a top plan view of a treatment system or equipment disclosed herein and configured/adapted for improving bacterial and/or microbial reduction and/or providing improved pathogen decontamination of vines, plants, and/or produce on at least one vine or in at least one farm field, in at least one embodiment of the present disclosure.

FIG. 5 illustrates a side plan view of at least one nozzle assembly and light units/panels comprising one or more UV-C light/radiation generating lamps, and an enlarged side plan view of the at least one nozzle assembly and at least one air knife assembly, in at least one embodiment of the present disclosure.

FIG. 6 illustrates a side plan view of a nozzle assembly of the treatment system or equipment disclosed herein having a plurality of nozzles, in at least one embodiment of the present disclosure.

FIG. 7 illustrates an end or rear plan view of a treatment system or equipment disclosed herein attached, connected, and/or coupled to a movable/drivable vehicle, in at least one embodiment of the present disclosure.

FIG. 8 illustrates a prospective side view of a movable/drivable vehicle attached, connected, and/or coupled to a treatment system or equipment disclosed herein that is configured/adapted for improving bacterial and/or microbial reduction and/or providing improved pathogen decontamination of vines, plants, and/or produce on at least one vine or in at least one farm field, in at least one embodiment of the present disclosure.

FIG. 9 illustrates a graph comprising 30-day area under the disease progress curve (hereinafter “AUDPC”) values analyzed by using Fishers least significant difference (hereinafter “LSD”) post, in an embodiment of the present disclosure.

FIG. 10 illustrates a graph comprising testing block layouts and testing results for rows of produce at a vineyard used during an in-field test plan, in an embodiment of the present disclosure.

FIG. 11 illustrates a side plan view of a movable/drivable vehicle attached, connected, and/or coupled to a treatment system or equipment disclosed herein, in at least one embodiment of the present disclosure.

FIG. 12 illustrates a perspective view of a movable/drivable vehicle attached, connected, and/or coupled to a movable/pullable treatment system or equipment disclosed herein, in at least one embodiment of the present disclosure.

FIG. 13 illustrates a side perspective view of a movable/pullable treatment system or equipment disclosed herein that is configured/adapted for improving bacterial and/or microbial reduction and/or providing improved pathogen decontamination of vines, plants, and/or produce on at least one vine or in at least one farm field, in at least one embodiment of the present disclosure.

FIG. 14 illustrates a bottom perspective view of a movable/pullable treatment system or equipment disclosed herein that is configured/adapted for improving bacterial and/or microbial reduction and/or providing improved pathogen decontamination of vines, plants, and/or produce on at least one vine or in at least one farm field, in at least one embodiment of the present disclosure.

### DETAILED DESCRIPTION OF THE DISCLOSURE

Illustrative examples of the subject matter claimed below will now be disclosed. In the interest of clarity, not all features of an actual implementation are described in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

Further, as used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

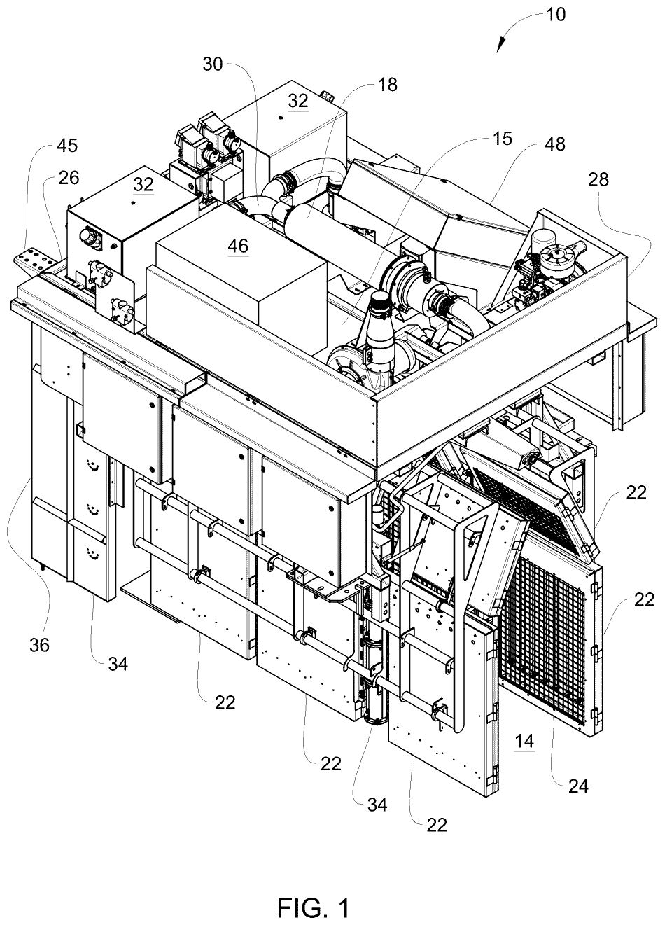

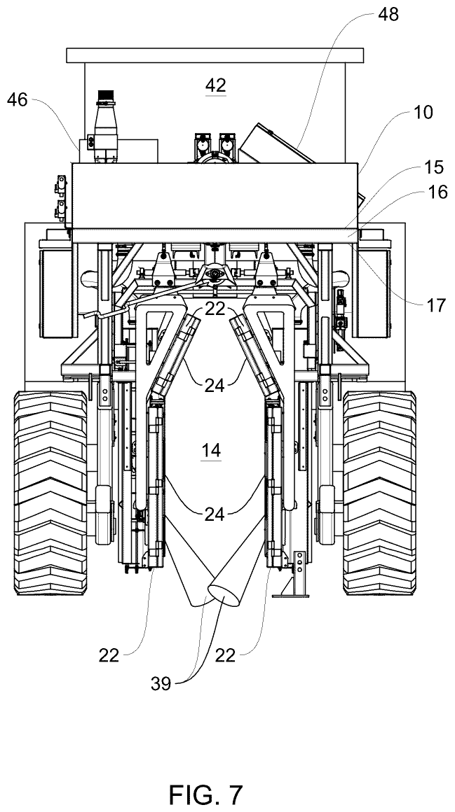

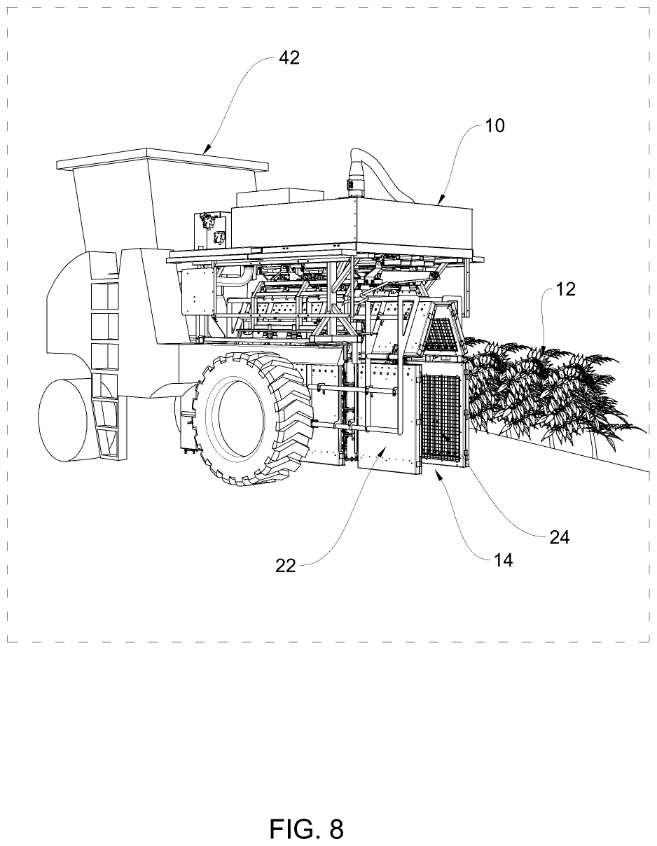

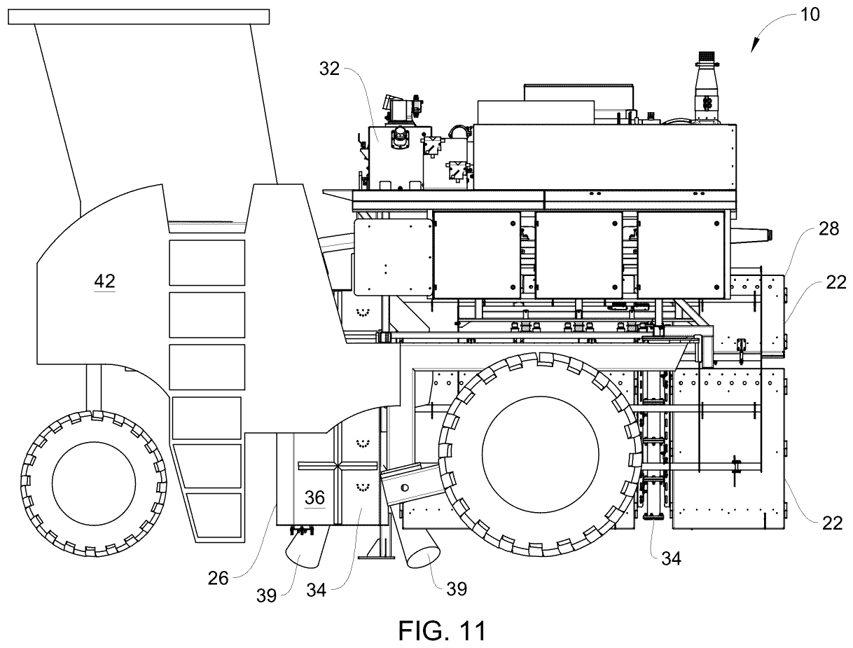

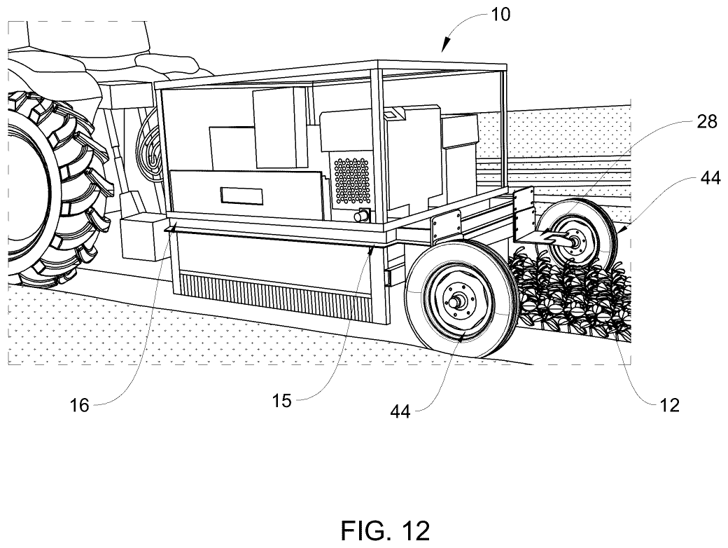

The present treatment systems and/or treatment equipment 10 disclosed herein (collectively referred to hereinafter as “the present system 10” as shown in FIGS. 1-4, 7, 8, and 11-14) are configured and/or adapted for treating one or more foods, one or more food products, one or more vines, one or more plants, and/or produce disposed or located on at least one vine or within at least one farm field (collectively referred to hereinafter as “the produce 12” as shown in FIGS. 8 and 12). The present system 10 and methods disclosed herein emit, generate, produce, and/or provide at least one hydroxyl-radical treatment (hereinafter “HR-treatment”) for treating the produce 12 such that improved bacterial and/or microbial reduction and/or improved pathogen decontamination of the produce 12 is achievable via the present system 10 and present methods. In other words, the HR-treatment may be generated, provided, or produced by, emitted from or by, and/or disposed within the present system 10 for treating the produce 12. In some embodiments, the present methods disclosed herein comprise, consist of, include, utilize, and/or use the present system 10 (collectively referred to hereinafter as “the present method”) to emit, expel, generate, produce, release, and/or provide one or more hydroxyl-radicals for the HR-treatment through and/or by introducing hydrogen peroxide mist, ozone gas, and/or UV-C light into a processing or treatment chamber 14 (hereinafter “chamber 14”) of the present system 10 as shown in FIGS. 1 and 7. The present system 10 and/or the present method disclosed herein may be improvements and/or comprise one or more improvements to the systems, equipment, and/or methods disclosed in U.S. Pat. No. 11,178,890 B2, issued Nov. 23, 2021, which is incorporated herein by reference in its entirety.

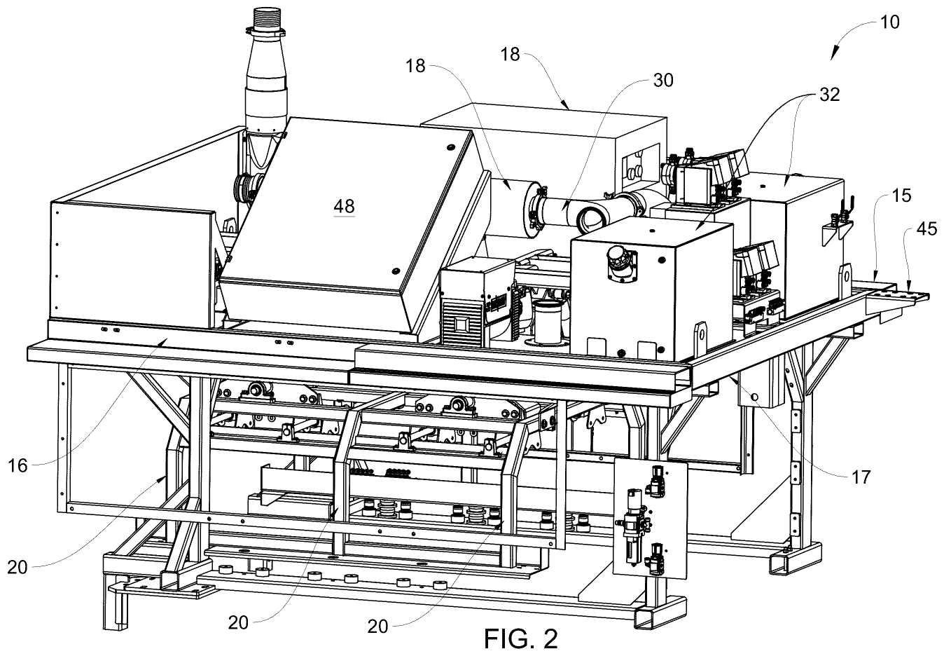

As shown in FIGS. 2, 4, 7, 13, and 14, the present system 10 may comprise, consist of, include, have, and/or contain a frame 16 with at least one ozone gas producing unit 18 (hereafter “ozone unit 18”) positioned, disposed, or located on a first side 15 of the frame 16, as shown in FIGS. 1, 2, and 4. As a result, the ozone unit 18 may be attached, connected, or coupled to, fastened to, and/or mounted onto the frame 16 or the first side 15 of the frame 16 for providing ozone gas to the present system 10 and/or the present method disclosed herein. Additionally, a circular, curved, rounded, or angled structure or cage 20 (collectively referred to hereinafter a “cage 20”) may be positioned, disposed, or located on a second opposite side 17 of the frame 16, as shown in FIG. 2. As a result, the cage 20 may be attached, connected, or coupled to and/or mounted onto the frame 16 or the second side 17 of the frame 16. In at least one embodiment, the chamber 14 may be defined by and/or within the frame 16, the cage 20, at least a portion of the frame 16, and/or at least a portion of the cage 20 of the present system 10. As a result, the frame 16 of the present system 10 may be disposed between the ozone unit 18 and the cage 20 and/or the chamber 14 of the present system 10. Moreover, the cage 20 may open or face away from the frame 16 and/or may be sized, shaped, configured, and/or adapted to receive the produce 12. As a result, the produce 12 may enter into, traverse through, exit out, and/or pass through the cage 20 when the present system 10 may move or may be moved along the produce 12 disposed within the field.

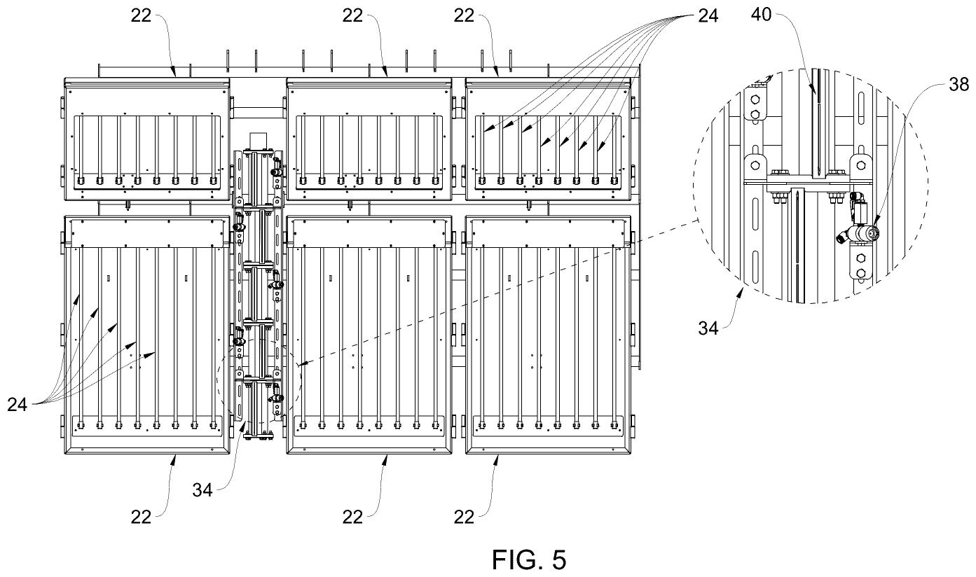

In some embodiments, the present system 10 may comprise, consist of, include, have and/or contain a plurality of UV-C light producing units 22 (hereinafter “light units 22”) as shown in FIGS. 1, 3, 5, 7, 8, and 11. Each of the light units 22 may comprise, consist of, include, have, and/or contain one or more UV-C light producing or emitting bulbs and/or lamps 24 (hereinafter “UVC lamps 24”) as shown in FIGS. 5, 7, 8, and 14. In at least one embodiment, the UVC lamps 24 may be, comprise, consist of, have, or include one or more germicidal lamps, one or more disinfection lamps, one or more sterilizer lamps, or a combination thereof. In some embodiments, the light units 22 may be in the form of one or more panels or sectional fixtures configured and/or adapted to produce, emit, and provide UV-C light or radiation to the chamber 14 of the present system 10 and/or the produce 12 during execution of the present method.

The UVC lamps 24 of the light units 22 may face towards or be directed into the chamber 14, may emit UV-C light or radiation into the chamber 14, may provide or deliver UV-C light or radiation to the chamber 14, and/or produce UV-C light or radiation within the chamber 14. Moreover, the chamber 14 may be defined by, within, and/or between the frame 16, the cage 20, the light units 22, the UVC lamps 24, or a combination thereof. Additionally, the frame 16 of the present system 10 may be positioned, disposed, and/or located between the ozone unit 18 and the chamber 14, the cage 20, the light units 22, the UVC lamps 24, or a combination thereof. In some embodiments, the light units 22 and/or the UVC lamps 24 may be attached to, connected to, coupled to, fastened to, and/or mounted on the cage 20, the frame 16, the second side 17 of the frame 16, or a combination thereof. As a result, the light units 22 and/or the UVC lamps 24 may be attached to, connected to, coupled to, fastened to, and/or mounted to the ozone unit 18 or the present system 10 via the frame 16, the second side 17 of the frame 16, the cage 20, or a combination thereof. In some embodiments, one or more UVC lamps of the UVC lamps 24 may have different sizes, shapes, lengths, widths, configurations, or a combination thereof. In at least one embodiment, the UVC lamps 24 of the present system may have at least one first length or at least one second length, wherein the at least one first length is greater than or less than the at least one second length as shown in FIGS. 1, 5, 7, 8, and 14.

In one or more embodiments, the light units 22 and/or the UVC lamps 24 may partitioned, separated, and/or divided into at least two horizontal rows and/or at least two vertical columns as shown in FIGS. 1, 3, 5, 7, 8, 11, and 25. As a result, a first horizontal row comprising a first plurality of light units 22 and/or UVC lamps 24 may be provided, positioned, located, and/or disposed above or below a second horizontal row comprising a second plurality of light units 22 and/or UVC lamps. In some embodiments, the present system 10 and/or the frame 16 may comprise, consist of, have, and/or include a first end 26 and a second end 28 that is located opposite with respect to the first end 26, as shown in FIGS. 1, 3, 4, 7, 11, 13, and 14. A first vertical column comprising the first plurality of light units 22 and/or UVC lamps 24 may be located, positioned, disposed or provided at the first end 26 or the second end 28, adjacent to the first end 26, or adjacent to the second end 28. In at least one embodiment, at least two first vertical columns of light units 22 and/or UVC lamps 24 may be located, positioned, disposed, or provided at or adjacent to the first end 26 and/or at least one second vertical column of light units 22 and/or UVC lamps 24 may be located, positioned, disposed, or provided at or adjacent to the second end 28. In one embodiment, the produce 12 to be treated by and/or treated by the present system 10 and/or present methods disclosed herein may enter the chamber 14 from the first end 26 and/or may exit the chamber 14 from the second end 28 and be exposed to the HR-treatment and/or UV-C light or radiation for a treatment time period or dwell time in the chamber 14 while the produce 12 is present or disposed within the chamber 14 and/or while the produce 12 passes through the chamber 14 from the first end 26 to the second end 28 of the present system 10, as shown in FIG. 12.

In one or more embodiments, one or more of the light units 22, the UVC lamps 24, and/or the UV-C light or radiation emitted from the light units 22 may be angled at one or more treatment angles with respect to each other, parallel with respect to each other, or a combination thereof as shown in FIGS. 1, 3, 5, 7, 8, 11, and 14. As a result, UV-C light or radiation emitted from or produced by the light units 22 and/or the UVC lamps 24 may be angled at the one or more treatment angles with respect to each other within the chamber 14. In some embodiments, the one or more treatment angles may consist of one or more angles that may be greater than about 0°, less than about 90°, greater than about 15°, less than about 75°, greater than about 30°, less than about 60°, about 30°, about 45°, and/or about 60°. In at least one embodiment, the one or more treatment angles may be in a range from greater than about 0° to less than about 90°.

In some embodiments, the ozone unit 18 may be, comprise, consist of, include, and/or contain one or ozone gas producing lamps (not shown in the drawings) which may provide, produce, manufacture, move, and/or generate ozone gas for the present system 10 from, based on, or consisting of air and/or oxygen supplied to the ozone unit 18. In an embodiment, the air and/or oxygen utilized by the ozone unit 18 may be air and/or oxygen present in the atmosphere surrounding the present system 10, the ozone unit 18, or a combination thereof. The air and/or oxygen present in the atmosphere may move into or enter the ozone unit 18 via one or more intake openings, orifices, or passages. The present system 10 and/or the ozone unit 18 may comprise, consist of, have, include, incorporate, and/or provide an ozone tube adapter and/or an ozone splitter 30 (collectively referred to hereinafter as “ozone splitter 30”), as shown in FIGS. 1, 2, and 4, that may be sized, shaped, configured, and/or adapted to deliver, move, or transport ozone gas from the ozone unit 18 to and/or into the chamber 14 of the present system 10. As a result, the ozone unit 18 may be connected to and in fluid communication with the chamber 14 via the ozone splitter 30 and/or ozone gas from the ozone unit 18 may be provided, transported, delivered, moved, and/or emitted into the chamber 14 via the ozone unit 18 and/or the ozone splitter 30.

As shown in FIGS. 1-4, and 11, the present system 10 and methods disclosed herein may comprise, consist of, have, include, and/or contain one or more mist producing units 32 (hereinafter “mist unit 32”) configured and/or adapted to provide, transport, deliver, move, emit, and/or produce at least one misting of hydrogen peroxide to or into the chamber 14 of the present system 10. Further, the mist unit 32 may be connected to and in fluid communication with the chamber 14 of the present system 10. As a result, hydrogen peroxide and/or at least one misting of hydrogen peroxide may be provided, transported, delivered, moved, emitted, and/or produced to/within the chamber 14 via the mist unit 32.

In at least one embodiment, the mist unit 32 or at least a portion of the mist unit 32 may be positioned, located, disposed, and/or provided on the frame 16 or on the first side 15 of the frame 16 that may be at, adjacent to, or near the ozone unit 18, the ozone splitter 30, or a combination thereof. In one embodiment, the ozone splitter 30 may be located, positioned, disposed, and/or provided between the ozone unit 18 and the mist unit 32 and/or on the first side 15 of the frame 16 that is opposite to the chamber 14, as shown in at least FIG. 1.

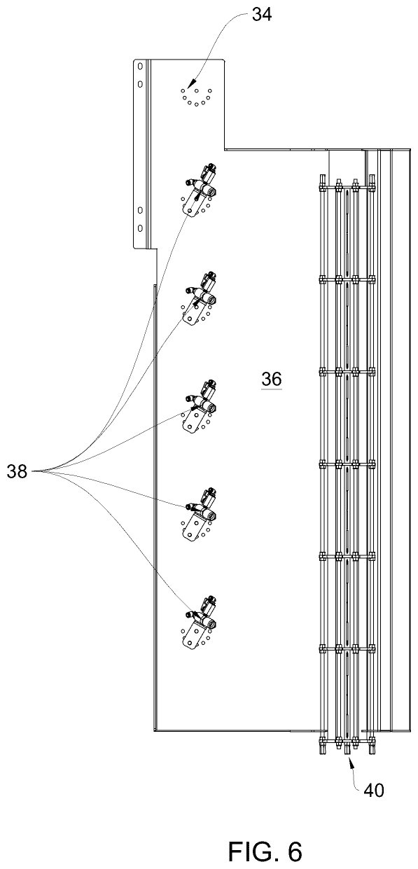

In one or more embodiments, the present system 10 may have, comprise, consist of, include, contain, and/or provide one or more nozzle assemblies 34 (hereinafter “nozzle assembly 34”), as shown in FIGS. 1, 3, 5, 11, 13, and 14. In some, embodiments, the nozzle assembly 34 may be located, disposed, positioned, and/or provided at, near, or adjacent to the first end 26 of the present system 10, the second end 28 of the present system 10, at one or more intermediate positions or locations between the first end 26 and the second end 28 of the present system 10, or a combination thereof as shown in FIGS. 1, 3, 5, 6, and 12-14. In some embodiments, the nozzle assembly 34 may be disposed, positioned, located, or provided at or adjacent to the first end 26 of the present system and/or at or adjacent to at least one intermediate position between the first end 26 and the second end 28 of the present system 10 as shown in FIGS. 1, 3, 5, and 11.

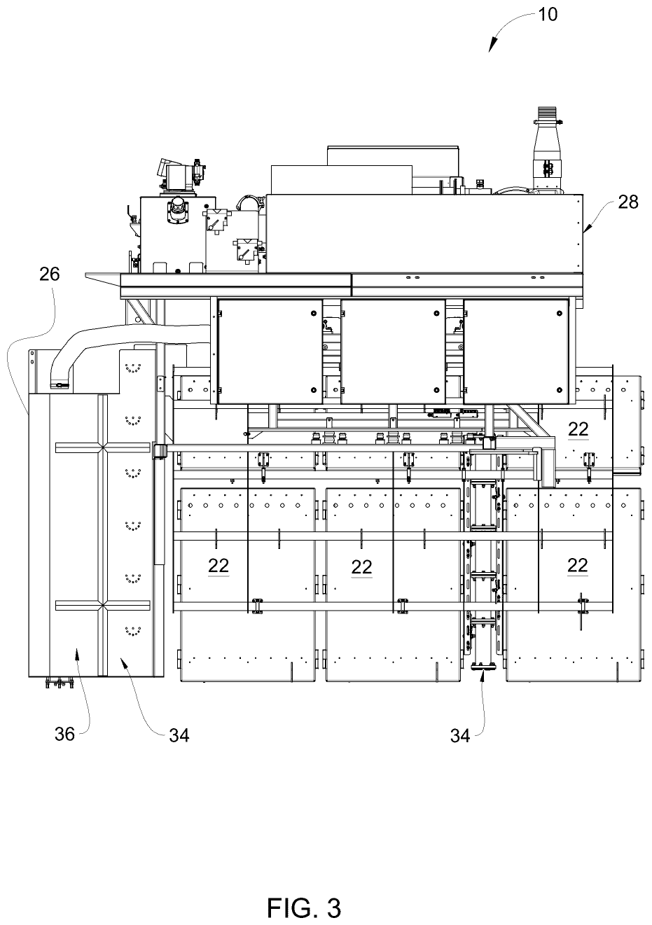

In some embodiments, the present system 10 may have, comprise, consist of, include, contain, and/or provide one or more side panels 36 (hereinafter “side panel 36”) which may comprise the nozzle assembly 34 as shown in FIGS. 1, 3, 6, and 11. In an embodiment, the nozzle assembly 34 may be integrally formed in or with the side panel 36 and/or the side panel 36 may be attached, connected, couples, fastened, secured, and/or fixed to the frame 16 of the present system 10 on one or both side of the present system 10 at, near, or adjacent to first end 26 of the present system 10. As a result, the nozzle assembly 34 of the side panel 36 may be positioned, disposed, located, provided at, near, or adjacent to the first end 26 of the present system 10 as shown in FIGS. 1 and 3. In at least one embodiment, the nozzle assembly 34 may be positioned, disposed, located, and/or provided between at least two different and/or adjacent light units 22, UVC lamps 24, or a combination thereof, as shown in FIGS. 1, 3, 5, and 11.

In some embodiments, the nozzle assembly 34 may have, comprise, consist of, include, contain, and/or provide a plurality of one or more spouts, outlets, jets, and/or nozzles 38 (hereinafter “nozzles 38”) as shown in FIGS. 5, 6, and 14. In one or more embodiments, the nozzles 38 and/or the nozzle assembly 34 may be sized, shaped, positioned, configured, aligned, and/or adapted such that the ozone gas and/or the hydrogen peroxide mist may enter or be injected or emitted into the chamber 14 for treating the produce 12 with the HR treatment while the produce 12 may be disposed within, moved into, or transversing the chamber 14. As a result, ozone gas from the ozone unit 18 and/or misting of hydrogen peroxide from the mist unit 32 may be mixed together, introduced or emitted into, and/or provided or disposed within the chamber 14 via the ozone unit 18, the cage 20, the ozone splitter 30, the mist unit 32, the nozzle assembly 34, the nozzles 38, or a combination thereof.

In one or more embodiments, the nozzles 38 may be sized, shaped, positioned, configured, angled, aligned, and/or adapted to adjust, change, control, direct, distribute, emit, inject, and/or modify one or more or a plurality of fluid flows and/or streams 39 comprising ozone gas and/or hydrogen peroxide mist (collectively referred to hereinafter as “stream 39”), as shown in FIGS. 7, 11, 13, and 14. As a result, the stream 39 may be delivered, directed, distributed, emitted, injected, introduce, moved, and/or transported into the chamber 14 from at least one of the ozone unit 18, the ozone splitter 30, the mist unit 32, the nozzle assembly 34, the side panel 36, the nozzles 38, or a combination thereof. The nozzles 38 may control one or more of the following parameters associated with the stream 39: a flow direction; a flow mass; a rate of flow; a flow shape; a flow speed; a flow pressure of the stream 39 that may exit the nozzles 38; or a combination thereof. In at least one embodiment, the stream 39 emitted, injected, and/or delivered into the chamber 14 may be in the form of at least one shape (i.e., the flow shape) and/or the at least one shape of the injection stream 39 may be, comprise, consist of, and/or include the shape of at least one cone. In some embodiments, the nozzles 38 may be, comprise, consist of, include, and/or contain at least one atomizer nozzle, at least one full cone nozzle, at least one jet nozzle, at least one spiral or swirl nozzle, at least one jet diffuser, at least one hollow cone nozzle, or a combination thereof.

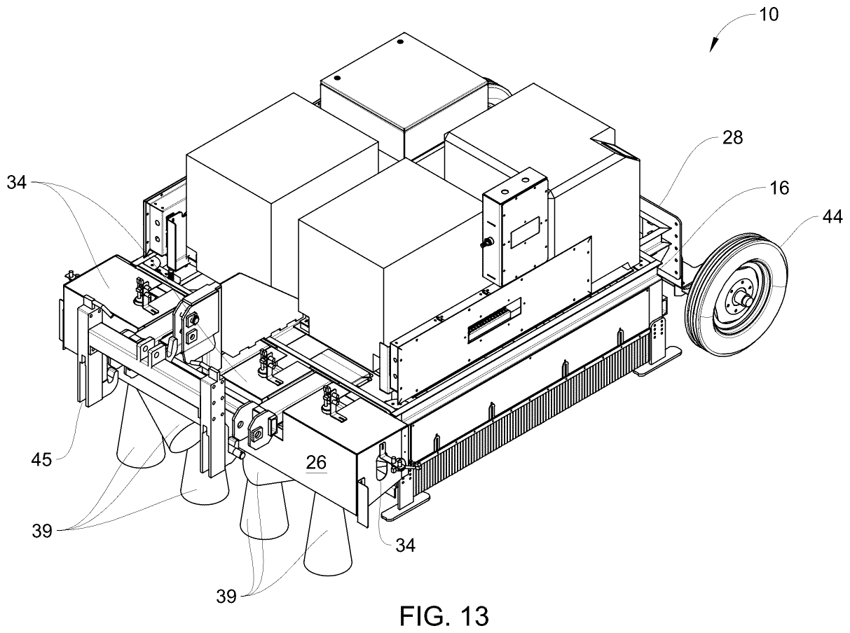

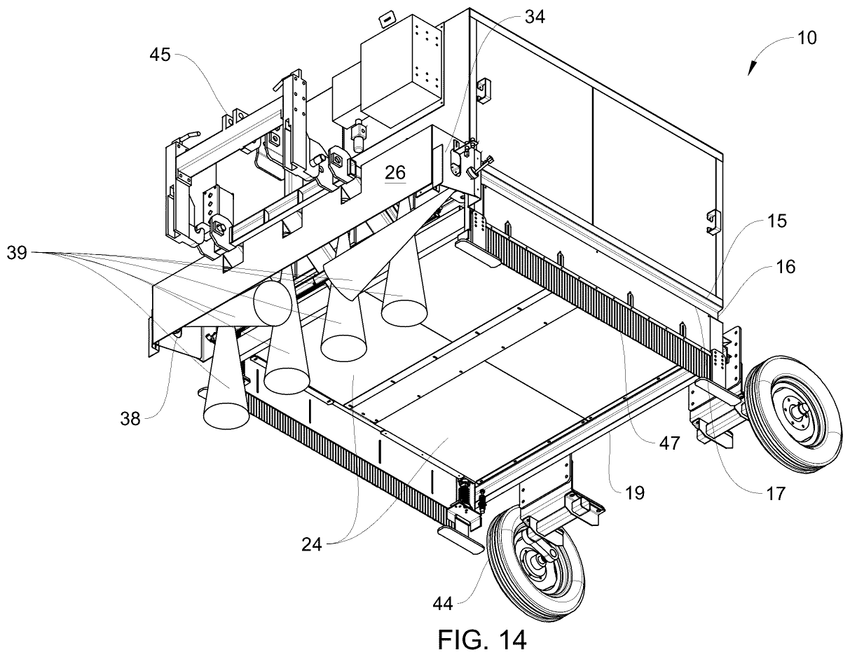

In some embodiments, the nozzles 38 may deliver, emit, inject, move, and/or transport the stream 39 vertically with respect to the field or ground that the present system 10 may traversing during the present method disclosed herein or in a direction that is perpendicular or substantially perpendicular to the longitudinal axis of the present system 10, wherein the longitudinal axis extends through the first and second ends 26, 28 of the present system 10, as shown in FIGS. 13 and 14. Additionally and/or alternatively, the nozzles 38 may deliver, emit, inject, move, and/or transport the stream 39 into the chamber 14 at one or more injection angles (see FIGS. 7, 11, 13, and 14) with respect to at least one longitudinal axis of at least one of the frame 16, the cage 20, the light units 22, the UVC lamps 24, the nozzle assembly 34, the side panel 36, or a combination thereof. In one or more embodiments, the one or more injection angles may be greater than about 0°, greater than about 15°, greater than about 30°, greater or less than about 45°, less than about 60°, less than about 75°, less than about 90°, about 30°, about 45°, and/or about 60°.

In some embodiments, the UV-C light or radiation emitted, produced, generated, or provided by the light units 22 and/or the UVC lamps 24 may generate, produce, provide, or form the one or more hydroxyl-radicals of the HR-treatment within the chamber 14 via the ozone gas, the one or more misting of hydrogen peroxide and/or the stream 39 disposed or distributed within and/or provided in the chamber 14. The HR-treatment comprising the one or more hydroxyl-radicals within the chamber 14 may treat the produce 12 as the produce 12 is present, positioned, located, and/or disposed within the chamber 14 of the present system 10 and/or while the produce 12 traverses the chamber 14 during the present method disclosed herein.

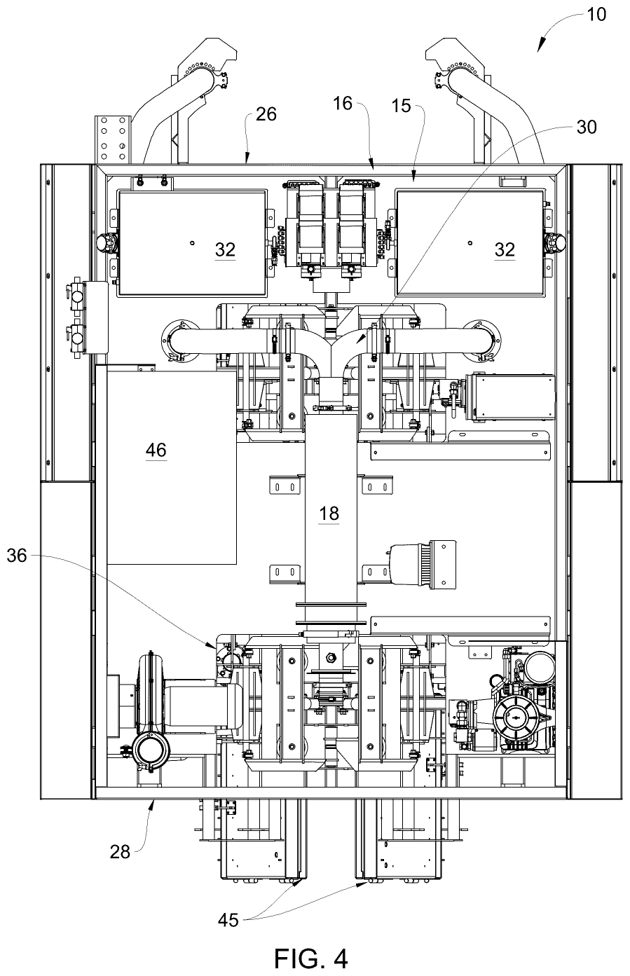

In one or more embodiments, the present system 10 may have, comprise, consist of, include, contain, and/or provide one or more air knife assemblies 40 (hereinafter “knife assembly 40”) which may mix the ozone gas and hydrogen peroxide mist together to form a treatment mixture prior to exposing the treatment mixture to the UV-C light or radiation for forming or providing the one or more hydroxyl-radicals of the HR-treatment. In some embodiments, the stream 39 may be, comprise, consist of, include, and/or contain the ozone gas, the hydrogen peroxide mist, or the treatment mixture. In at least one embodiment, the knife assembly 40 injects, moves, pushes, or propels the ozone gas, the hydrogen peroxide mist, the treatment mixture, and/or the stream 39 into the chamber 14 of the present system 10 prior to or during the formation and/or production of the one or more hydroxyl-radicals of the HR-treatment. As a result, the ozone gas, the hydrogen peroxide mist, the treatment mixture, and/or the stream 39 may be moved, transported, injected, emitted, delivered, distributed, and/or provided into or within the chamber 14 via the nozzle assembly 34, the nozzles 38, the knife assembly 40, or a combination thereof. In some embodiments, the knife assembly 40 may be attached, connect, coupled, fastened, mounted, and/or affixed to or onto the frame 16, the cage 20, the nozzle assembly 34, the side panel 36, and/or the nozzles 38 such that the knife assembly 40 may be positioned, located, disposed, and/or provided at, near, or adjacent to the second side 17 of the frame 16, the cage 20, the first end 26 of the present system 10, the second end 28 of the present system 10, at least one intermediate position between the first end 26 and the second end 28 of the present system, or a combination thereof, as shown in FIGS. 4-6.

In some embodiments, the produce 12 disclosed herein and to be treated with the HR-treatment may be grown and/or maintained on at least one vine and/or in at least one farm field, as shown in FIGS. 8 and 12. When the produce 12 is grown and/or maintained on at least one vine and/or in at least one farm field and the present system 10 or the present method treats the produce 12 with the HR-treatment, then such HR-treatment may be referred to as on-the-vine HR-treatment (hereinafter “OTV treatment”) and/or the present system 10 may referred to as an on-the-vine treatment unit (hereinafter “the OTV treatment unit 10”). Hereinafter, the terms “HR-treatment” and “OTV treatment” and the terms “the present system 10” and “the OTV treatment unit 10”) may be used interchangeably and/or may comprise one or more of the same, similar, or substantially similar components, elements, parameters, structural features/relationships, and/or method steps and/or method sub-steps.

In some embodiments, the produce 12 treated with or to be treated with the HR-treatment disclosed herein may be, comprise, consist of, include, and/or contain at least one agricultural food product selected from the group consisting of: cereals and/or grains; fruits; vegetables; legumes; nuts and/or seeds, herbs and/or spices; and a combination thereof. In some embodiments, the fruits and vegetables may be selected from the group consisting of: apples; bananas; oranges; grapes; strawberries; mangoes; avocados; tomatoes; lemons; peaches; pears; pineapples; watermelons; blueberries; kiwis; cherries; cranberries; papayas; raspberries; blackberries; potatoes; carrots; broccoli; spinach; lettuce; cucumbers; bell peppers; onions; zucchini; cauliflower; eggplants; green beans; peas; asparagus; brussels sprouts; celery; corn; radishes; beets; squash; amaranth; buckwheat; dragon fruit; lychee; durian; plantains; jackfruit; and a combination thereof. In other embodiments, the produce 12 may comprise, consist of, include, and/or contain at least one plant, at least one vine, at least one plant that grows at least one vine, or a combination thereof. Additionally, the at least one vine may comprise, consist of, include, and/or contain at least one plant with a growth habit of trailing or scandent stems, lianas, runners, or a combination thereof. Moreover, the produce 12 may be, comprise, consist of, include, and/or contain one or more food products that are fresh and/or one or more farm-produced crops, foods, or products, such as, for example, fruits, grains, oats, vegetables, or a combination thereof.

In some embodiments, the present system 10 may be in the form of a pushable, movable, and/or drivable unit as shown in FIGS. 7, 8 and 11, a pullable and drivable attachment as shown in FIGS. 12-14, or a combination thereof. In at least one embodiment, the present system 10 may be, comprise, consist of, include, contain and/or provide a movable/drivable unit or a pullable/drivable attachment that may be attached, connected, coupled, fastened, mounted, secured, and/or affixed to movable, drivable, and/or motorized agricultural equipment and/or vehicle 42 (collectively referred to hereinafter as “vehicle 42”) as shown in FIGS. 7, 8, 11, and 12. In at least one embodiment, the vehicle 42 may be selected from the group consisting of: tractors; harvesters; combines; plows; seeder/planters; cultivators; harrows; sprayers; fertilizer spreaders; balers; tillage equipment; mowers; wagons and/or carts; GPS and/or precision farming equipment; all-terrain vehicles; rugged/rough terrain vehicles; utility vehicles; and a combination thereof. In some embodiments, the vehicle 42 may be, comprise, consist of, include, and/or contain at least one tractor selected from the group consisting of: a farm tractor, a garden tractor, a utility tractor, a compact tractor, a wheeled tractor, a track tractor, a specialty crop tractor, and a combination thereof. In at least one embodiment, the vehicle 42 may be, comprise, consist of, include, and/or contain: an orchard tractor; a vineyard tractor; a forage harvester; a grape harvester; a crop sprayer; an orchard sprayer; a front-end loader; a GPS-guided tractor; an autonomous tractor; an all-terrain vehicle; a utility task vehicle; a row cleaner; or a combination thereof. In one embodiment, the vehicle 42 may at least one known farming vehicle and the present system 10 may be utilized alone with the vehicle 42 or in combination with one or more tractor or agricultural implements and/or the one or more tractor or agricultural implements may be selected from the group consisting of plows; harrows; fertilizer spreaders; balers; wagons or trailers; orchard cabs; other tractor attachments; or a combination thereof. In one or more embodiments, the present system 10 disclosed herein may be in the form of or may comprise at least one tractor attachment, such as, for example, a front loader, a backhoe, a bucket, a fork; a blade; a sprayer; a rotary cutter; a mower; trenchers, a farm trailer, or a combination thereof.

In one or more embodiments, the present system 10 may be in the form of or may be, comprise, consist of, and/or include at least one tractor attachment comprising a vehicle propulsion system 44 (hereinafter “propulsion system 44”) as shown in FIG. 12-14. In some embodiments, the propulsion system 44 may be disposed, located, positioned at, near, or adjacent to the second end 28 of the system 10, the second side 17 of the frame 16, or a combination thereof. Additionally, the propulsion system 44 may be attached, connected, coupled, fastened, and/or mounted to the frame 16, the second end 28 of the system 10, the first side 15 of the frame 16, the second side 17 of the frame 16, or a combination thereof. The propulsion system 44 of the present system 10 may be, have, comprise, consist of, and/or include one or more plates, one or more belts, one or more tracks, one or more wheels, one or more tires, or a combination thereof.

In some embodiments, the present system 10, when in the form of a tractor attachment, may comprise one or more fasteners 45, as shown in FIGS. 1, 2, 13 and 14. The one or more fasteners 45 may be disposed, located, positioned at, near, or adjacent to the first end 26 of the system 10, the first side 15 of the frame 16, the second side 17 of the frame 16, or a combination thereof. The one or more fasteners 45 may be attached, connected, coupled, fastened, and/or mounted to the frame 16, the first end 26 of the system 10, the first side 15 of the frame 16, the second side 17 of the frame 16, or a combination thereof. In some embodiments, the one or more fasteners 45 of the present system 10 may be sized, shaped, configured, adapted, and/or designed to affix, attach, connect, couple, fasten, hitch, hook, link, and/or secure the present system 10 to the vehicle 42 as shown in FIG. 12. In one embodiment, the one or more fasteners 45 may be a known fastener capable of attaching, connecting, coupling, hitching, hooking, or linking the present system 10 to the vehicle 12.

In one or more embodiments, the present system 10 may comprise one or more dividers and/or separators 47 (hereinafter “dividers 47”) as shown in FIGS. 12-14. The dividers 47 may be disposed, located, positioned, mounted, and/or affixed to a perimeter of the chamber 14, at least one side of the perimeter of the chamber 14, at least one portion of the perimeter of the chamber 14, or a combination thereof. In some embodiments, the dividers 47 may be affixed, attached, connected, coupled, fastened, mounted, and/or secured to the frame 16, the cage 20, the first side 15 of the frame 16, the second side 17 of the frame 16, the light units 22, UVC lamps 24, the nozzle assembly 34, the side panel 36, or a combination thereof. As a result, the dividers 47 partitioned the chamber 14 away from the atmosphere and/or environment surrounding or outside the present system 10, chamber 14, the cage 20, or a combination thereof. Additionally, the dividers 47 may block, obscure, prevent, and/or hinder air drafts, dust, light, rain, sunshine, water, other environmental elements, or a combination thereof from entering or being introduced into the chamber 14. In one or more embodiments, the dividers 47 may be, comprise, consist of, include, and/or contain one or more natural and/or synthetic curtains, drapes, or blinds, one or more natural and/or synthetic cloth materials, one or more natural and/or synthetic fibers, one or more natural and/or synthetic polymers, one or more natural and/or synthetic fringes, one or more natural and/or synthetic divider strips, one or more strip curtains, or a combination thereof.

As shown in FIGS. 1, 2, 4, and 7, the present system 10 may comprise at least one generator 46 that is configured and/or adapted for powering and/or operating at least one selected from the ozone unit 18, the light units 22, the UVC lamps 24, the mist unit 32, the nozzle assembly 34, the knife assembly 40, the vehicle propulsion system 44, at least one controller 48, or a combination thereof. In at least one embodiment, the at least one controller 48 may be configured and/or adapted for controlling, activating, deactivating, operating, changing, adjusting, and/or manipulating at least one selected from the ozone unit 18, the light units 22, the UVC lamps 24, the mist unit 32, the nozzle assembly 34, the knife assembly 40, the vehicle propulsion system 44, at least one controller 48, or a combination thereof. The at least one generator 46 and/or the at least one controller 48 may be in communication with the ozone unit 18, the light units 22, the UVC lamps 24, the mist unit 32, the nozzle assembly 34, the knife assembly 40, the vehicle propulsion system 44, at least one controller 48, or a combination thereof.

In one or more embodiments, the present system 10 and/or the present method disclosed herein may combine at least one UV-treatment (i.e., UV-C light and/or radiation) with one or more oxidizing agents such as, for example, the ozone gas, the hydrogen peroxide mist, the treatment mixture, or the steam 39. The one or more oxidizing agents may be degraded by UV photons of the UV-treatment, the light units 22, and/or the UVC lamps 24 to form, produce, and/or provide one or more short-lived, antimicrobial hydroxyl-radicals of the HR-treatment that may penetrate into one or more areas on one or more surfaces of the produce 12. As a result of the penetration by the one or more short-lived antimicrobial hydroxy-radicals, bacterial and/or microbial reduction and/or pathogen decontamination of the produce 12 may be achieved by the present system 10, the present method, the HR-treatment, and/or the OTV-treatment disclosed herein. Moreover, decontamination of the produce 12 may be achievable by the present system 10, the present method, the HR-treatment, and/or the OTV-treatment disclosed herein. In at least one embodiment, one or more important, relevant, or critical parameters for the present system 10 and/or the present method may be, comprise, consist of, and/or include: one or more temperatures associated with the present system 10 and/or the produce 12; one or more hydrogen peroxide concentrations associated with the chamber 14 and/or the stream 39; one or more hydrogen peroxide volume flow rates into the chamber 14; one or more ozone flow rates into the chamber 12; one or more UV radiation rates into the chamber 12 and/or emitted from the UVC lamps 24; one or more dwell times of the produce 12 within the chamber 14 and/or being exposed to HR-treatment or OTV-treatment; or a combination thereof. In at least one embodiment, the reaction to generate or for generating the hydroxyl-radicals of the HR-treatment in the chamber 14 may be favored at a temperature of about 48° C. or less and/or above the typical room temperature of about 22° C. Moreover, the important, relevant, or critical parameters of the present system 10 may be or comprise one or more parameters associated with the ozone unit 18, the light units 22, the UVC lamps 24, the mist unit 32, the nozzle assembly 34, the nozzles 38, the stream 39, the knife assembly 40, the vehicle propulsion system 44, at least one controller 48, or a combination thereof.

In some embodiments, the present systems 10 may comprise, consist of, include, or contain the chamber 14 which is operably connected to one or more means for producing and/or suppling each of UV-C light or radiation via the light units 22 and/or the UVC lamps 24, hydrogen peroxide mist via the mist unit 32, ozone gas via the ozone unit 18, the stream 39 via the nozzles 48, and/or heat via a heating unit (not shown in the drawings) during the HR-treatment or the OTV treatment of the produce 12. In at least one embodiment, the present system 10 and/or the present method may utilize or use ozone gas in the chamber 14 and/or even introduce ozonated water containing peroxide into the chamber 14 during the HR-treatment or the OTV treatment of the produce 12. In an embodiment, the vehicle 42 may transport or move the present system 10 over, above, around and/or along the produce 12 on the at least one vine or in the at least one field while the present system 10 may emit, produce, and/or provide the HR-treatment or the OTV treatment to the produce 12. In further embodiments, the present system 10 and/or the present method disclosed herein may be configured/adapted for removal of pesticide from the produce 12 during the HR-treatment or OTV treatment of the produce 12.

In one or more embodiments, the hydrogen peroxide mist from the mist unit 32 and/or the stream 39 may comprise hydrogen peroxide at a concentration from about 0.1% to about 10%, from about 0.25% to about 7.5%, from about 0.5% to about 5%, or from about 1% to about 3%, wherein all concentration values are weight percentages based on the total weight of the hydrogen peroxide mist. In some embodiments, the wavelength emitted by the UVC lamps 24 may range from about 220 nm to about 280 nm, from about 230 nm to about 270 nm, from about 240 nm to about 265 nm, or from about 250 nm to about 260 nm. In an embodiment, the wavelength emitted by the UVC lamps 24 of the light units 22 may be about 254 nm.

In some embodiments, the chamber 14 of the present system 10 and/or the present method may be kept or maintained at a predetermined humidity during the HR-treatment or the OTV treatment, wherein the predetermined humidity may be from about 40% to about 100%, from about 50% to 100%, from about 60 to about 100%, or from about 50% to about 90%. In another embodiment, the present system 10 and/or the present method may be configured and/or may comprise or utilize parameters such that the produce 12 may have one or more dwell times in the chamber 14 of from about 5 seconds to about 2 minutes. In embodiments utilizing the vehicle 42, the dwell time of the produce 12 within the chamber 14 may be from about 1 second to about 15 seconds, from about 2 seconds to about 12 seconds, from about 3 seconds to about 10 seconds, or from about 4 seconds to about 7 seconds. Additionally, the vehicle 42 may achieve, exhibit, facilitate, and/or have at least one tracker speed with respect to the produce 12 ranging from about 0.5 kilometer/hour (hereinafter “km/hr”) to about 7.5 km/hr, from about 1 km/hr to about 5 km/hr, or from about 1.5 km/hr to about 3 hm/hr.

In an embodiment, one or more parameters of the present method executable via the present system 10 disclosed herein may be as follows:

UV light parameter of the UVC lamps 24: The total wattage (hereinafter “W”) may be greater than about 0 W up to about 10,000 W. For example, the UVC lamps 24 of the light units 22 may be configured as follows: up to about 400×UV-C 23 W lamps emitting at about 254 nm (wavelength range between about 290 nm and about 100 nm); or 3×17 W=about 51 W total, or 4 bulbs×46 W+2 bulbs on sides×34 W per bulb=about 252 W total.

Ozone parameter of the ozone unit 18: up to about 400×ozone light bulbs emitting at about 174 nm (wavelength range±about 50%) within the ozone unit 18. About 95% of the ultraviolet energy emitted from the germicidal lamps used by the present system 10 and/or present method is at the mercury resonance wavelength of about 254 nanometers. This wavelength is in the region of maximum germicidal effectiveness and is highly lethal to virus, bacteria, protozoa and mold. Ultraviolet wavelengths shorter than about 200 nanometers are capable of producing ozone from oxygen (O2) in the air present within the ozone unit 18 during the HR-treatment of the produce 12. The ozone lamps (not shown in the drawings) contained, disposed, housed, located, and/or positioned within the ozone unit 18, in addition to emitting germicidal ultraviolet output at about 254 nanometer wavelength, may also emit ozone-producing radiation or rays at about 185 nanometer wavelength.

Although only about 7% of the total energy output of the ozone lamps of the ozone unit 18 are about 185 nanometer wavelength, this wavelength energy has the unique capability to destroy organics by oxidation of the organics to carbon dioxide gas. The extent of the microbial reduction is dependent on both ultraviolet purifier sizing (expressed as dosage level units in microwatt-seconds per square centimeter).

Distance of the produce 12 from the light units 22, the UVC lamps 24, the nozzle assembly 34, the nozzles 38, the stream 39, and/or the air knife assembly 40 may be from about 1 cm to about 200 cm.

In some embodiments, hydrogen peroxide concentration of hydrogen peroxide mist, the treatment mixture, and/or the stream 39 within the chamber 14 may be from about 1% to about 12% volume/volume aqueous solution, preferably from about 1 to about 6%, and more preferably from about 2 to about 4% or about 3%.

The hydrogen peroxide flow rate into the chamber 14 may be, for example, from about 0 or about 0.01 to about 10 liters per minute. In one or more embodiments, the hydrogen peroxide flow rate (at about 3% hydrogen peroxide concentration) may be from about 22.5 to about 37.5 liters per hour, from about 25 to about 35 liters per hour, from about 27.5 to about 32.5 liters per hour, or about 30 liters per hour.

The ozone gas flow rate of ozone gas within the chamber 14 may be from about 540 to about 900 g/hr, from about 580 to about 860 g/hr, from about 620 to about 820 g/hr, from about 660 to about 780 g/hr, from about 700 to about 740 g/hr, or about 720 g/hr.

UV exposure rate of UV-C light and/or radiation within the chamber 14 may be from about 9.75 to about 16.25 mW/cm², from about 11 to about 15 mW/cm², from about 12 to about 14 mW/cm², or about 13 mW/cm².

Temperature in the chamber 14 may be at or maintained between about 22° C. and about 60° C., from about 40 to about 55° C., or about 48° C.

Humidity inside the chamber 14 may be between about 60% to about 100%.

In embodiments, wherein the present system 10 may be, comprise, consist of, include, or contain a manually pushable or pullable unit, the present system 10 may distribute, emit, produce, or provide HP-treatment or OTV-treatment (i.e., the hydrogen peroxide mist, the ozone gas, the treatment mixture, the stream 39, and/or the UVC light or radiation emitted from the UVC lamps 24) onto the produce 12 as the present system 10 may pass on top and/or over the produce 12 and/or while the present system 10 may move above or along the produce 12 in the field.

Experiment 1 as set forth herein was conducted with the manually pushable unit disclosed herein comprising the present system 10 and the results are as follows:

### Experiment 1: Efficacy on Powdery Mildew Severity (9 Weeks)

50 grapevine seedlings of a susceptible cultivar of interest in 1-gallon pots were the subject of Experiment 1.

Seedlings were grown in a hoop house for one month.

Grapevines were inoculated on a weekly basis, as explained below, for a total of 5 weeks.

Treatments were as follows:
plants were non-inoculated and non-treated;
plants were non-inoculated and treated with the hydroxyl-radical treatment disclosed herein;
plants were non-inoculated and treated with fungicide;
plants were inoculated weekly with powdery mildew at “a low concentration” and non-treated; plants were weekly inoculated with powdery mildew at “a low concentration” and treated with the hydroxyl-radical treatment disclosed herein; and plants were inoculated weekly with powdery mildew at a “high concentration” and treated with the hydroxyl-radical treatment.

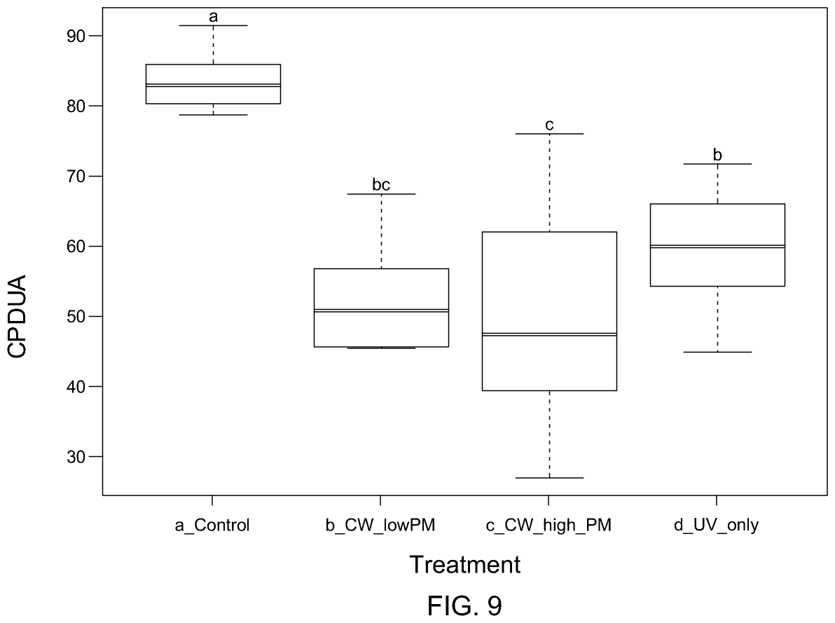

These treated plants were rated for signs and symptoms of powdery mildew periodically for the length of the experiment. For Experiment 1, the 30-day AUDPC values were analyzed using Fishers LSD post as shown in FIG. 9.

Under high disease pressure, the HR-treatment disclosed herein achieves improved performance when directly compared to UV-treatment alone.

Experiment 2 utilized the movable and pushable unit or unit attachment (i.e., the present system 10) that had the configuration set forth in FIG. 8.

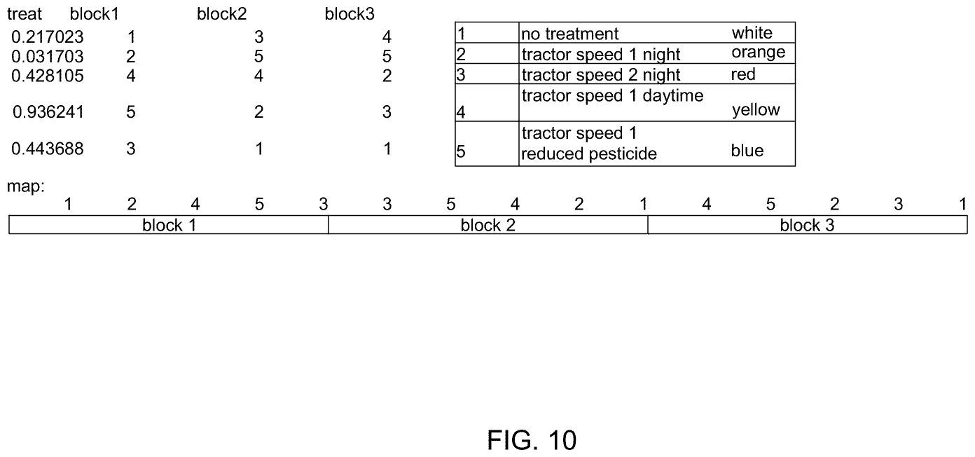

Experiment 2 comprises an in-field test and the test results of Experiment 2 are set forth in FIG. 10 which shows testing block layouts and testing results for rows at testing vineyard.

In more than one embodiment, the present system 10 disclosed may have one or more or a combination of the configuration(s) and/or structural feature(s)/relationship(s) as shown in FIGS. 1-8 and 11-14. In some embodiments, the movable unit or attachment unit disclosed herein (i.e., the present system 10) may be configured as or in the form of a tow attachment which may have one or more or a combination of the configuration(s) and/or structural feature(s)/relationship(s) as shown in FIGS. 12-14.

### Experiment 3: Treatment of Baby and Mature Spinach Using the Clean Tow System

To determine the reduction of E. coli and shelf-life of baby and mature spinach when treated with the Clean Tow system (i.e., the present system 10).

### Method

E. coli K12 was cultivated in TSB overnight at 3700. The culture was diluted 1:10 with distilled water and sprayed over the spinach plants. The inoculum was given at least 30 min to attach prior to applying the Clean Tote treatment (i.e., the HR-treatment disclosed herein). The plants were treated by the unit (i.e., the present system 10) being passed over the inoculated plants at different speeds (i.e., dose) in an embodiment of the present method.

The plants were cut using a knife at the right, center and left of the row then returned to the laboratory. Samples (25 g) were suspended in 225 ml of saline then stomached for 60 s. A dilution series was prepared in saline and plated onto MacConkey agar that was incubated at 37° C. for 24 h. The results of Experiment 3 are as follows:

| | | | Log Count |
|---|---|---|---|
| Treatment | CfU/g | Average SD | Reduction |
| Baby Spinach | | | |
| Control | | | |
| A | 5.26 | 5.58 ± 0.35 | |
| B | 6.05 | | |
| C | 5.61 | | |
| D | 5.76 | | |
| E | 5.20 | | |
| 0.5 mph | | | |
| A | 4.70 | 4.96 ± 0.34 | 0.62 |
| B | 5.34 | | |
| C | 4.85 | | |
| 1.0 mph | | | |
| A | 5.71 | 5.92 ± 0.19 | 0 |
| B | 6.09 | | |
| C | 5.95 | | |
| 2.0 mph | | | |
| A | 4.78 | 5.18 ± 0.38 | 0.40 |
| B | 5.20 | | |
| C | 5.54 | | |
| 3.5 mph | | | |
| A | 6.04 | 6.25 ± 0.26 | 0 |
| B | 6.16 | | |
| C | 6.54 | | |
| 4.5 mph | | | |
| A | 5.51 | 5.85 ± 00.39 | 0 |
| B | 5.78 | | |
| C | 6.28 | | |

| | | | Log Count |
|---|---|---|---|
| Treatment | CfU/g | Average SD | Reduction |
| Mature Spinach | | | |
| Control | | | |
| A | 6.24 | 6.31 ± 0.13 | |
| B | 6.40 | | |
| C | 6.38 | | |
| D | 6.11 | | |
| E | 6.41 | | |
| 0.5 mph | | | |
| A | 5.77 | 5.26 ± 0.97 | 1.05 |
| B | 5.86 | | |
| C | 4.14 | | |
| 1.0 mph | | | |
| A | 5.64 | 5.63 ± 0.66 | 0.68 |
| B | 6.28 | | |
| C | 4.95 | | |
| 2.0 mph | | | |
| A | 6.29 | 5.60 ± 0.73 | 0.71 |
| B | 5.65 | | |
| C | 4.85 | | |
| 3.5 mph | | | |
| A | 6.01 | 5.48 ± 0.36 | 0.53 |
| B | 5.36 | | |
| C | 5.96 | | |
| 4.5 mph | | | |
| A | 4.47 | 4.97 ± 0.93 | 1.34 |
| B | 6.03 | | |
| C | 4.39 | | |

### Experiment 4: Treatment of Baby and Mature Spinach Using the Clean Tow System

To determine the reduction of E. coli and shelf-life of baby and mature spinach when treated with the Clean Tow System (i.e., the present system 10). The aim of the Tow testing was to conduct the same test as previously and to compare the result differences and observe if there is a change in efficacy.

### Method

The following trials conducted on the Clean Tow System are as follows:

| Trial Number | Description |
|---|---|
| 1 | Control |
| 2 | All Ingredients on, electrostatic on, 1 mph speed |
| 3 | All ingredients on, electrostatic on, 3.5 mph speed |
| 4 | All ingredients on, electrostatic on, 4.5 mph speed |
| 5 | All ingredients on, electrostatic off, 1 mph speed |
| 6 | All ingredients on, electrostatic off, 3.5 mph speed |
| 7 | All ingredients on, electrostatic off, 4.5 mph speed |

E. coli K12 was used as the inoculant to mimic the same methods as last year. The inoculant was sprayed onto designated sections in the field, and the tractor reached the appropriate speed pertaining to the trial number to apply the correct treatment. Results were collected by UofG and sent back to the lab for analysis.

Additionally, a shelf-life study was conducted by UofG. Samples from each trial were be collected from non-inoculated patches of spinach to observe the effect of the treatment on the shelf life of the spinach.

### Experiment 5: Treatment of Baby and Mature Spinach Using the Clean Flow System

To perform a test on the naturally occurring bacteria levels on the spinach when treated by the Clean Flow System (i.e., present system 10). This was done using TAC petrifilms so that no foreign contaminants were introduced into the post-harvest Clean Works equipment.

### Method

Control samples were collected after the spinach was harvested from the grading table.

These samples will be the control. Ideally, these treated samples were collected with a normal throughput of spinach passing through the Clean Flow System to mimic standard operating conditions. The treated samples were collected after the spinach has passed through the Clean Flow equipment. The samples were collected and analyzed by UofG. Samples were also collected by UofG to perform a shelf-life analysis between the control and treated spinach.

The final phase of testing was conducted via a shelf-life analysis by combining the Clean Tow treatment with the Clean Flow treatment. To summarize, the following shelf-life trials were conducted:

| Trial Number | Description |
|---|---|
| 1 | Control |
| 2 | Treat spinach in field with Tow, |
| | collect shelf-life samples |
| 3 | Treat spinach post-harvest with Flow, |
| | collect shelf-life samples |
| 4 | Treat spinach in the field with Tow, |
| | Harvest, Treat with Flow, Collect samples |

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Obviously, many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

What is claimed is:

1. A system configure for treating produce on the vine or in the field with a hydroxyl-radical treatment, the system comprises:
a movable frame defining a processing chamber between a first end of the movable frame and a second end of the movable frame that is opposite with respect to the first end of the movable frame;
at least one mist producing unit disposed connected to the movable frame and in fluid communication with the processing chamber for introducing hydrogen peroxide mist into the processing chamber;
one or more UV-C lamps connected to the movable frame and disposed within or adjacent to the processing chamber for emitting UV-C light and/or radiation into the processing chamber; and
at least one ozone gas producing unit connected to the movable frame and in fluid communication with the processing chamber for introducing ozone gas into the processing chamber,
wherein the processing chamber is movable over and/or along the produce on the vine or in the field and antimicrobial hydroxy-radicals of the hydroxyl-radical treatment are formed via the hydrogen peroxide, UV-C light and/or radiation, and the ozone gas within the processing chamber such that bacterial and/or microbial reduction and/or pathogen decontamination of the produce is achievable via the antimicrobial hydroxy-radicals of the hydroxyl-radical treatment emitted within the processing chamber.

2. The system of claim 1, further comprising:
a cage connected to the movable frame, wherein the processing chamber is further defined by one or more surfaces of the cage.

3. The system of claim 2, further comprising:
one or more light panels comprising the one or more UV-C lamps and connected to the movable frame by the cage.

4. The system of claim 1, further comprising:
a first nozzle assembly connected to the movable frame at or adjacent to the first end of the movable frame such that nozzles of the first nozzle assembly are directed towards the processing chamber.

5. The system of claim 4, further comprising:
A second nozzle assembly connected to the movable frame at an intermediate position or location between the first and second ends of the movable frame such that nozzles of the second nozzle assembly are directed towards the processing chamber.

6. The system of claim 5, wherein at least one of the at least one mist producing unit and the at least one ozone gas producing unit are in fluid communication with the processing chamber via at least one of the first nozzle assembly and the second nozzle assembly.

7. The system of claim 1, wherein the one or more UV-C lamps comprises first UV-C lamps and second UV-C lamps and the second UV-C lamps are angled at an angle with respect to the first UV-C lamps.

8. The system of claim 7, wherein the angle is greater than about 0° and less than about 90°.

9. The system of claim 8, wherein the angle ranges from about 30° to about 60°.

10. The system of claim 1, further comprising:
at least one air knife assembly connected to the movable frame, in fluid communication with the processing chamber, and configured to mix the ozone gas and the hydrogen peroxide mist to provide a treatment mixture within the processing chamber.

11. The system of claim 10, further comprising:
at least one nozzle assembly connected to the movable frame, disposed adjacent to the at least one air knife assembly, and in fluid communication with the at least one mist producing unit for introducing the hydrogen peroxide mist into the processing chamber.

12. Treatment equipment comprising:
a movable vehicle; and
the system of claim 1 connected or coupled to the movable vehicle.

13. The treatment equipment of claim 12, wherein the movable vehicle is selected from the group consisting of a tractor, a harvester, a tow, a trailer, and a wagon.

14. The treatment equipment of claim 12, wherein the system further comprises a vehicle propulsion system connected to the movable frame and configured for towing system with the movable vehicle.

15. A method comprising:
providing one or more antimicrobial hydroxyl-radicals of the hydroxyl-radical treatment by exposing the hydrogen peroxide mist and/or ozone gas within the process chamber to UV-C light and/or radiation emitted by the one or more UV-C lamps; and
treating produce on a vine or in a field with the one or more hydroxyl-radicals of the hydroxyl-radical treatment by moving the system of claim 1 over, above, and/or around the produce on the vine or in the field.

16. The method of claim 15, wherein the hydrogen peroxide mist comprises hydrogen peroxide at a concentration from about 0.1% to about 10%, based on a total weight of the hydrogen peroxide mist.

17. The method of claim 16, wherein a wavelength emitted by the one or more UV-C lamps ranges from about 220 nm to about 280 nm.

18. The method of claim 17, wherein the wavelength emitted by the one or more UV-C lamps is about 254 nm.

19. The method of claim 15, wherein the produce are present within the processing chamber for a dwell time from about 1 second to about 15 seconds.

20. The method of claim 15, wherein the system has a tracker speed with respect to the produce on the vine or in the field ranging from about 0.5 km/hr to about 7.5 km/hr.
